SEQUENCE LISTING (4 sequences)

SEQ ID NO: 1 - DNA, 72 bp, synthetic construct
  source 1..72: mol_type = other DNA
gtctttaattgcatgaaaagctgtaaagcagtcaaaaggatatcagcgagtgtgtaggctggagctgcttcg

SEQ ID NO: 2 - DNA, 70 bp, synthetic construct
  source 1..70: mol_type = other DNA
cgccccctaagaacggagcgtgcgagtttccccgcactccgctcaagcctcatatgaatatcctccttag

SEQ ID NO: 3 - DNA, 70 bp, synthetic construct
  source 1..70: mol_type = other DNA
aaagcttttatagtgagggacttcagaaataccctagaaaaggaactgttcatatgaatatcctccttag

SEQ ID NO: 4 - DNA, 72 bp, synthetic construct
  source 1..72: mol_type = other DNA
ccgccaggcccactctccggtcgtggccgccttaaggacggttccgttcttgtgtaggctggagctgcttcg

(19) United States
(12) Patent Application Publication
(10) Pub. No.: US 2025/0051783 A1
(43) Pub. Date: Feb. 13, 2025

(54) METHOD FOR INHIBITING GROWTH OF TYPE I FIMBRIAE OF ESCHERICHIA COLI

(71) Applicant: Sichuan University, Chengdu (CN)

(72) Inventors: Hongning WANG, Chengdu (CN); Hongcheng WEI, Chengdu (CN); Changwei LEI, Chengdu (CN); Zhonghan LI, Chengdu (CN); Wei XU, Chengdu (CN)

(21) Appl. No.: 18/889,307

(22) Filed: Sep. 18, 2024

(51) Int. Cl. C12N 15/70; C12N 1/20

(52) U.S. Cl. CPC C12N 15/70; C12N 1/20; C12N 2800/101

(57) ABSTRACT

A protein capable of inhibiting type I fimbriae of Escherichia coli (E. coli) is identified, where the protein is named as Type I fimbrial repressor (TIFR) protein, and is encoded by a TIfr gene with an ID of GW576_23830. A method is further provided for inhibiting the growth of type I fimbriae of an E. coli strain, in which the TIfr gene is transformed into the strain.

### REFERENCE TO AN ELECTRONIC SEQUENCE LISTING

The contents of the electronic sequence listing (Name: SequenceListing.xml; Size: 10,898 bytes; and Date of Creation: Oct. 16, 2024) is herein incorporated by reference in its entirety.

### TECHNICAL FIELD

This application relates to inhibition of type I fimbriae of Escherichia coli (E. coli), and more specifically to a method for inhibiting growth of type I fimbriae of E. coli.

### BACKGROUND

Escherichia coli (E. coli) is a gram-negative brevibacterium. As an opportunistic pathogen, E. coli can cause gastrointestinal infections in humans or animals (pigs and chickens are the most susceptible hosts), and induce urinary tract infections, arthritis, meningitis, and septicemia-type infections, which is harmful to human health and economic development. The main virulence factors of E. coli include specific fimbria antigens and pathogenic toxins, among which the type I fimbria, as an adhesin antigen of E. coli, is an important virulence determinant that mediate the binding of bacteria to the D-mannose receptor of eukaryotic cells on the surface of E. coli to colonize the bacteria on the mucosa, thus triggering infections. The type-I fimbriae also play an important role in the process of bacterial invasion on cells, mediating the invasion of various cells by pathogenic E. coli. In addition, the type-I fimbriae can promote the bacterial adsorption on biotic or abiotic surfaces, which is an important factor in biofilm formation, thereby significantly increasing bacterial drug resistance. The synthesis and assembly of the type I fimbriae of E. coli require at least nine genes (FimB, FimZ, FimA, FimI, FimC, FimD, FimF, FimG and FimH). Among these genes, FimA is the major structural protein of the type I fimbriae, which forms a complex protein with FimF, FimG, and FimH. The presence or absence of fimbriae is called “phase transition”, and the “phase transition switch” is controlled by an inverted DNA sequence (a length of 314 bp) containing the FimA promoter. The “phase transition switch” is regulated by two genes (i.e., FimB and FimE) upstream of FimA. FimC is required for the synthesis of fimbriae proteins. FimD controls the localization of fimbriae in the cell membrane. FimF, FimG, and FimH regulate the length and number of fimbriae, and FimH is a key factor for regulating the binding of fimbriae to the D-mannose receptor of eukaryotic cells.

Although the function and pathogenicity of the type I fimbriae are increasingly reported, there are still many unknown factors that regulate the phase transition of the type I fimbriae. To deal with the problem of drug resistance caused by the formation of biofilm by bacteria, some metal nanomaterials have been developed. As for the formed biofilm, the physical removal means is effective. However, as for the prevention of the formation of biofilm, the use of biological factors to block the adhesion process mediated by fimbriae may be the most effective way. The proteins found in this application can inhibit type-I fimbriae of E. coli, which provides an important reference for solving the above problems.

### SUMMARY

In order to break the bottleneck in the current researches about biological factor-mediated inhibition of fimbria synthesis, the present disclosure provides a newly-identified protein capable of inhibiting the growth of type I fimbriae of Escherichia coli (E. coli) and an inhibiting method based on the same. The new protein can inhibit the expression of type I fimbrial gene clusters, thereby stably suppressing the synthesis of type I fimbriae of E. coli and providing a basis for controlling the fimbrial adhesion-mediated bacterial virulence and the formation of the bacterial biofilms.

To realize the above purpose of stably inhibiting the synthesis of type I fimbriae of E. coli by inhibiting the expression of the type I fimbrial gene clusters, the present disclosure provides the following technical solutions.

In a first aspect, this application provides a Type I fimbrial repressor (TIFR) protein, wherein the TIFR protein is encoded by gene GW576_23830.

In a second aspect, this application provides an identification method of the aforementioned TIFR protein, comprising:
(1) acquiring a pEC51 plasmid carrying ItrA gene (gene ID: GW576_23335, SEQ ID NO: 5) and TIfr gene (gene ID: GW576_23830, SEQ ID NO: 6) through isolation and identification;
(2) transforming the pEC51 plasmid into an E. coli C600 (EC600) strain followed by culture with a solid medium containing 8 μg/ml of Mueller-Hinton agar (MHA) to screen and name a successfully transformed strain as a CFII strain;
(3) subjecting a wild-type EC600 strain and the CFII strain to streak culture overnight on a MHA solid medium; picking an monoclone of the EC600 strain and an monoclone of the CFII strain followed by respectively culture in 50 mL of a LB medium under shaking at 37° C. and 180 r/min for 8 h, where the transcriptome sequencing results show that read counts of Nove100011, Nove100012, and AOF44210.1 are relatively larger, and all of them are corresponding to the ItrA gene;
(4) knocking the ItrA gene out from the pEC51 plasmid by Red homologous recombination through steps of:
   designing a pair of homologous recombination primers (F: GTCTTTAATTGCATGAAAAGCTGTAAAGCAGTCAAAAGGATATCAGCG AGTGTGTAGGCTGGAGCTGCTTCG (SEQ ID NO: 1)/R:CGCCCCCTAAGAACGGAGCGTGCGAGTTTCCCCGCACTCCGCTCA AGCCTCATATGAATATCCTCCTTAG (SEQ ID NO: 2)); performing amplification using a pKD3 plasmid as a template to obtain an amplified product; subjecting the amplified product to gel purification to obtain a chloramphenicol-resistant gene containing a homology arm; simultaneously introducing a pKD46-Tetracycline (Tet) plasmid and the chloramphenicol-resistant gene containing the homology arm into the CFII stain by electrotransformation followed by screening with a MHA solid medium containing 16 μg/mL chloramphenicol; picking a monoclone followed by amplification in a LB liquid medium containing 16 μg/mL chloramphenicol; and inoculating the ItrA-knockout strain (named as CFIIΔItrA strain) into a liquid medium followed by shaking culture at 42° C. to eliminate the pKD46-Tet plasmid;
(5) culturing the CFIIΔItrA strain according to step (3) followed by transcriptome analysis, where transcriptome sequencing results of the CFII strain and the CFIIΔItrA strain show that gene expression levels of FimA, FimC, FimD, FimF, FimG, FimH, FimI of the CFIIΔItrA strain are significantly reduced;
(6) respectively inoculating the EC600 strain, the CFII strain and the CFIIΔItrA strain into the MHA solid medium followed by culture at 37° C. overnight; respectively picking a monoclone of the EC600 strain, a monoclone of the CFII strain and a monoclone of the CFIIΔItrA for culture overnight in the LB liquid medium under shaking at 37° C., followed by inoculation into the MHA solid medium for streak culture overnight; respectively picking monoclones of the EC600 strain, the CFII strain and the CFIIΔItrA strain followed by staining with 2% phosphotungstic acid for 3-10 s, air-drying at room temperature and observation under a JEM-1400FLASH transmission electron microscope, where the CFIIΔItrA strain is free of fimbriae;
(7) constructing a pUC19 plasmid containing the ItrA gene; and transforming the pUC19 plasmid into the CFIIΔItrA strain, where the fimbrial growth is restored in the CFIIΔItrA strain through the complementation of the ItrA gene;
(8) as evidenced by the transcriptome sequencing that a hypothetical protein (i.e., the TIfr protein) is highly expressed in the CFIIΔItrA strain, knocking out the TIfr gene by Red homologous recombination (F:AAAGCTTTTATAGTGAGGGACTTCAGAAATACCCTAGAAAAGGAACTGT TCATATGAATATCCTCCTTAG (SEQ ID NO: 3)/R:CCGCCAGGCCCACTCTCCGGTCGTGGCCGCCTTAAGGACGGTTCCGTT CTTGTGTAGGCTGGAGCTGCTTCG (SEQ ID NO: 4)), where the fimbrial growth is restored after the TIfr gene is knocked out, indicating that the protein encoded by the gene GW576_23830 can inhibit the expression of type I fimbriae; and the protein is named as Type I fimbrial repressor (TIFR) protein, and the corresponding gene GW576_23830 is named as TIfr, and the GW576_23830-knockout strain is ΔItrAΔTIfr strain;
constructing a pUC19 plasmid containing the TIfr gene; and transforming the pUC19 plasmid into the ΔItrAΔTIfr strain, where the fimbriae are suppressed again through the complementation of the TIfr gene; and
(9) transforming the pUC19 plasmid containing the TIfr gene into a LF82 strain, where it is found that exogenous expression of the TIFR protein can effectively inhibit the type I fimbriae of the LF82 strain.

In a third aspect, this application provides a method for inhibiting growth of type I fimbriae of an E. coli strain, comprising:
transforming the TIfr gene into the E. coli strain, wherein an ID of the TIfr gene is GW576_23830.

Compared with the prior art, the present disclosure has at least the following beneficial effects.

In this application, the ItrA gene in the IncFII plasmid (i.e., the pEC51 plasmid) is knocked out to construct an E. coli strain without fimbriae. Furthermore, it is found that the hypothetical protein (gene ID: GW576_23830, named TIFR) can effectively inhibit the generation of type I fimbriae of E. coli, providing reference for reducing the virulence of E. coli and biofilm formation, and controlling the pathogenicity and drug resistance of E. coli.

### BRIEF DESCRIPTION OF THE DRAWINGS

FIG. 1 schematically shows identification of a TIfr gene that encodes a TIFR protein to inhibit the production of type I fimbriae of Escherichia coli (E. coli) according to an embodiment of the present disclosure;

FIG. 2 schematically shows a pEC51 plasmid constructed according to an embodiment of the present disclosure;

FIGS. 3-4 schematically show screening of a CFII strain according to an embodiment of the present disclosure, where the plasmid is transformed into an EC600 strain, and the screening is performed on a solid medium containing 8 μL/mL Mueller-Hinton agar (MHA);

FIG. 5 schematically shows step (3) of the identification method according to an embodiment of the present disclosure;

FIG. 6 schematically shows step (4) of the identification method according to an embodiment of the present disclosure;

FIG. 7 schematically shows transformation and verification of a pKD46 plasmid according to an embodiment of the present disclosure;

FIG. 8 schematically shows elimination of the pKD46 plasmid according to an embodiment of the present disclosure;

FIG. 9 schematically shows step (5) of the identification method according to an embodiment of the present disclosure;

FIG. 10 schematically shows step (6) of the identification method according to an embodiment of the present disclosure;

FIGS. 11-13 schematically shows step (7) of the identification method according to an embodiment of the present disclosure;

FIGS. 14-17 schematically shows step (8) of the identification method according to an embodiment of the present disclosure; and

FIGS. 18-19 schematically shows step (9) of the identification method according to an embodiment of the present disclosure.

### DETAILED DESCRIPTION OF EMBODIMENTS

The technical solutions of the present disclosure will be described clearly and completely below with reference to the embodiments. Obviously, described herein are merely some embodiments of the present disclosure, instead of all embodiments. Based on these embodiments, all other embodiments obtained by one of ordinary skill in the art without making creative effort shall fall within the scope of the present disclosure.

### Experimental Example 1 Discovery and Identification of a New Protein Capable of Inhibiting Type I Fimbriae of E. coli

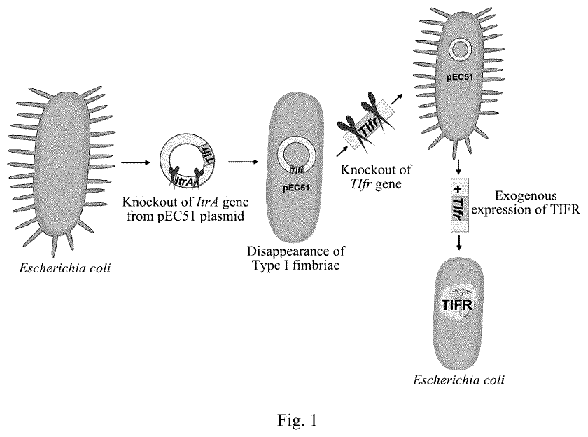

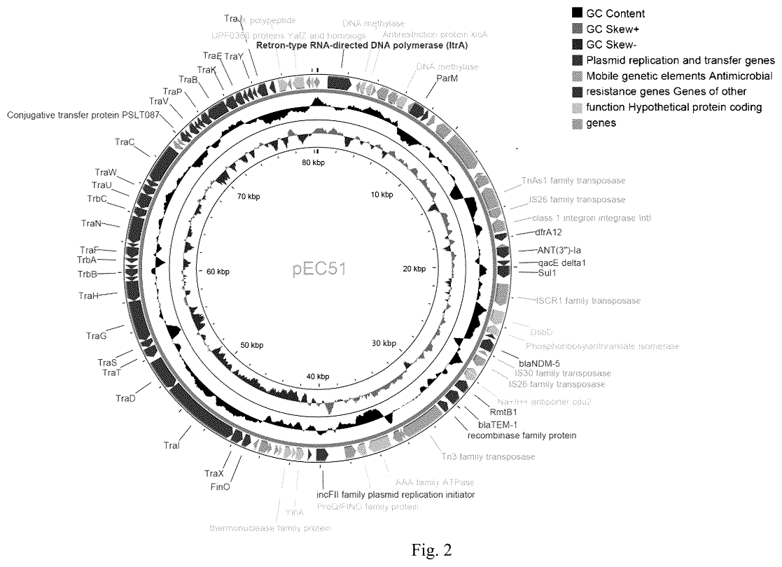

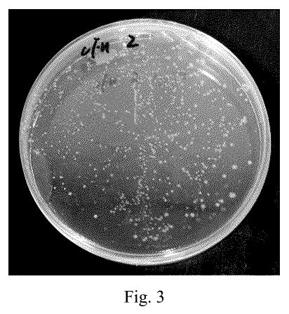

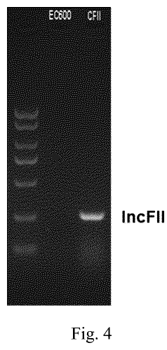

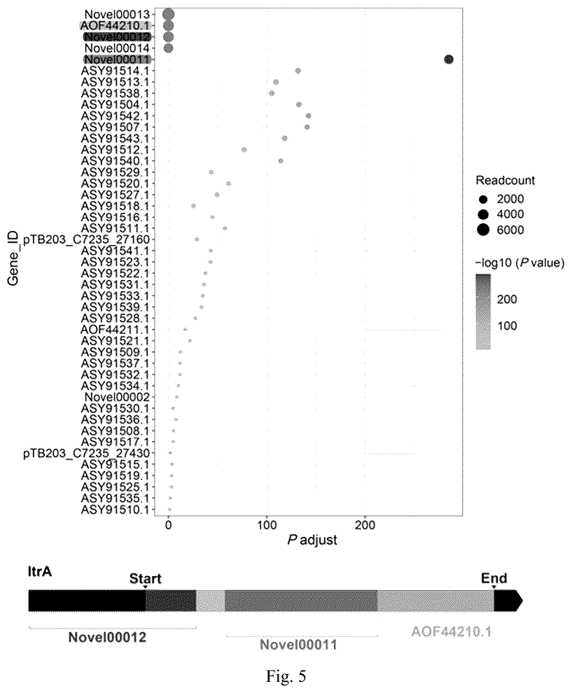

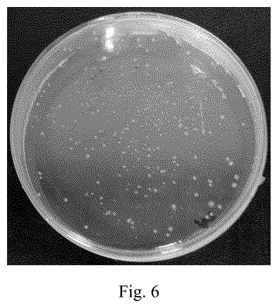

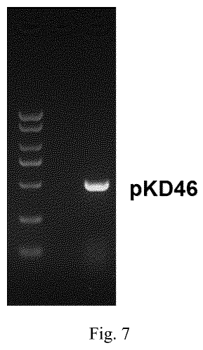

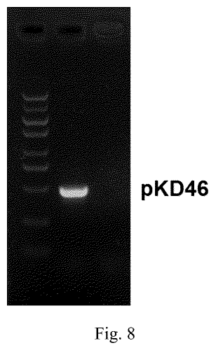

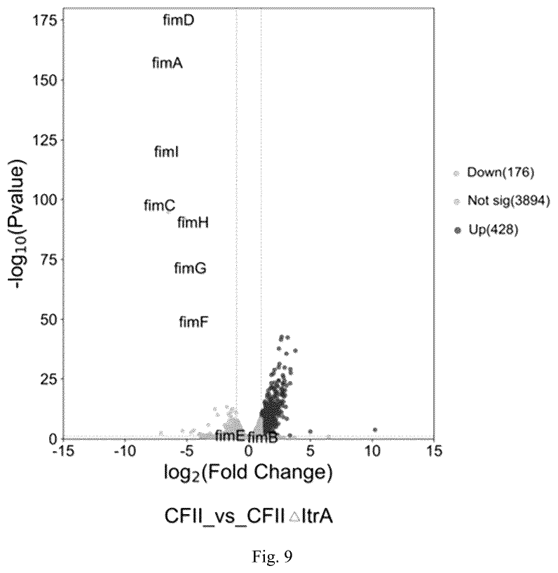

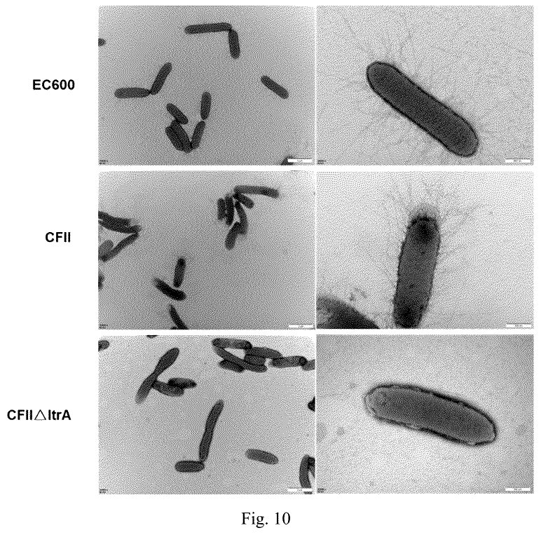

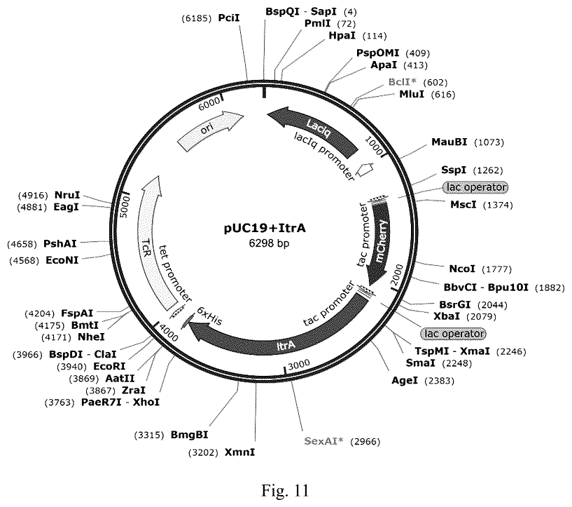

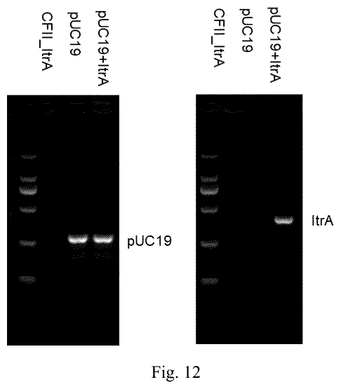

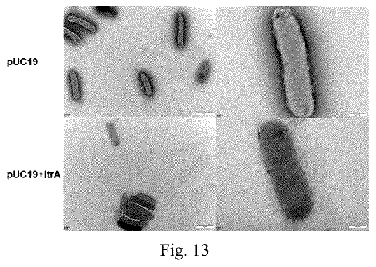

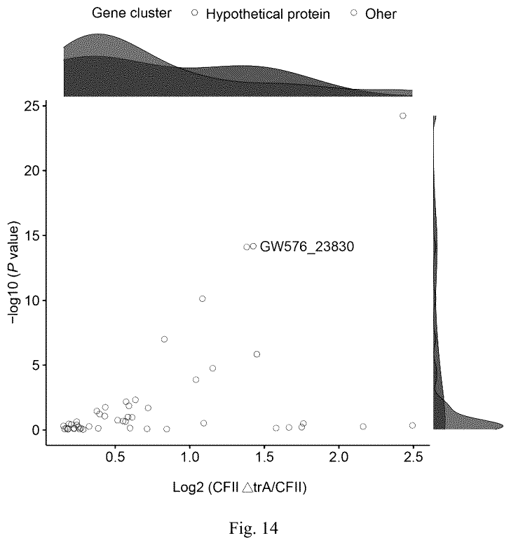

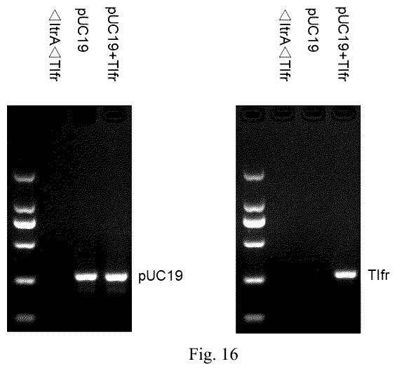

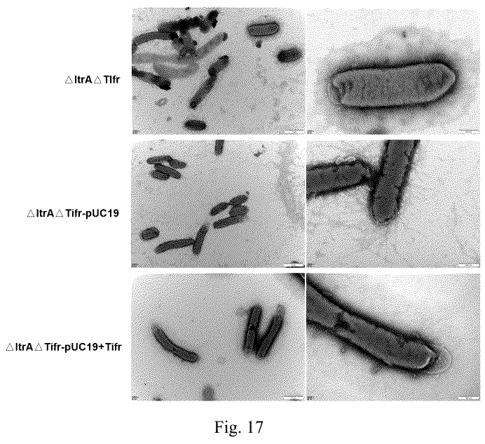

The new protein capable of inhibiting type I fimbriae of E. coli was discovered and identified as follows (referring to FIGS. 1-19).

(1) A pEC51 plasmid carrying ItrA gene (gene ID: GW576_23335, SEQ ID NO: 5) and TIfr gene (gene ID: GW576_23830, SEQ ID NO: 6) is acquired through isolation and identification.

(2) The pEC51 plasmid is transformed into an E. coli C600 (EC600) strain followed by culture with a solid medium containing 8 μg/ml of Mueller-Hinton agar (MHA) to screen and name a successfully transformed strain as a CFII strain.

(3) A wild-type EC600 strain and the CFII strain are subjected to streak culture overnight on a MHA solid medium. An monoclone of the EC600 strain and an monoclone of the CFII strain are picked followed by respectively culture in 50 ml of a LB medium under shaking at 37° C. and 180 r/min for 8 h, where the transcriptome sequencing results show that read counts of Nove100011, Nove100012, and AOF44210.1 are relatively larger, and all of them are corresponding to the ItrA gene (Retron-type RNA-directed DNA polymerase).

(4) The ItrA gene is knocked out from the pEC51 plasmid by Red homologous recombination through the following steps.

A pair of homologous recombination primers is designed (F: GTCTTTAATTGCATGAAAAGCTGTAAAGCAGTCAAAAGGATATCAGCGAGT GTGTAGGCTGGAGCTGCTTCG (SEQ ID NO: 1)/R: CGCCCCCTAAGAACGGAGCGTGCGAGTTTCCCCGCACTCCGCTCAAGCCT CATATGAATATCCTCCTTAG (SEQ ID NO: 2)). Amplification is performed using a pKD3 plasmid as a template to obtain an amplified product. The amplified product is subjected to gel purification to obtain a chloramphenicol-resistant gene containing a homology arm. The pKD46-Tet plasmid and a purified DNA product are simultaneously introduced into the CFII stain electrotransformation followed by screening with a MHA solid medium containing 16 μg/mL chloramphenicol. A monoclone is picked followed by amplification in a LB liquid medium containing 16 μg/mL chloramphenicol. The ItrA-knockout strain (named as CFIIΔItrA strain) is inoculated into a liquid medium followed by shaking culture at 42° C. to eliminate the pKD46-Tet plasmid.

(5) The CFIIΔItrA strain is cultured according to step (3) followed by transcriptome analysis, where transcriptome sequencing results of the CFII strain and the CFIIΔItrA strain show that gene expression levels of FimA, FimC, FimD, FimF, FimG, FimH, FimI of the CFIIΔItrA strain are significantly reduced.

(6) The EC600 strain, the CFII strain and the CFIIΔItrA strain are respectively inoculated into the MHA solid medium followed by culture at 37° C. overnight. A monoclone of the EC600 strain, a monoclone of the CFII strain and a monoclone of the CFIIΔItrA are respectively picked for culture overnight in the LB liquid medium under shaking at 37° C., followed by inoculation into the MHA solid medium for streak culture overnight. Monoclones of the EC600 strain, the CFII strain and the CFIIΔItrA strain are respectively picked, followed by staining with 2% phosphotungstic acid for 3-10 s, air-drying at room temperature and observation under a JEM-1400FLASH transmission electron microscope, where the CFIIΔItrA strain is free of fimbriae.

(7) A pUC19 plasmid containing the ItrA gene is constructed and transformed into the CFIIΔItrA strain, where the fimbrial growth is restored in the CFIIΔItrA strain through the complementation of the ItrA gene.

(8) As evidenced by the transcriptome sequencing analysis that a hypothetical protein (i.e., the TIfr protein) is highly expressed in the CFIIΔItrA strain, the TIfr gene is knocked out by Red homologous recombination (F:AAAGCTTTTATAGTGAGGGACTTCAGAAATACCCTAGAAAAGGAACTGTTC ATATGAATATCCTCCTTAG (SEQ ID NO: 3)/R: CCGCCAGGCCCACTCTCCGGTCGTGGCCGCCTTAAGGACGGTTCCGTTCTT GTGTAGGCTGGAGCTGCTTCG (SEQ ID NO: 4)), indicating that the protein encoded by the gene GW576_23830 can inhibit the expression of type-I fimbriae, and the protein is named as Type I fimbrial repressor (TIFR) protein, and the corresponding gene GW576_23830 is named as TIfr, and the GW576_23830-knockout strain is ΔItrAΔTIfr strain. A pUC19 plasmid containing the TIfr gene is constructed and transformed into the ΔItrAΔTIfr strain, where the fimbriae are suppressed again through the complementation of the TIfr gene.

### Example 1

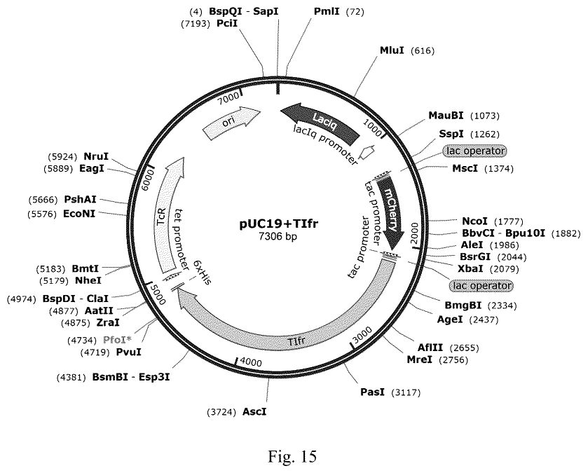

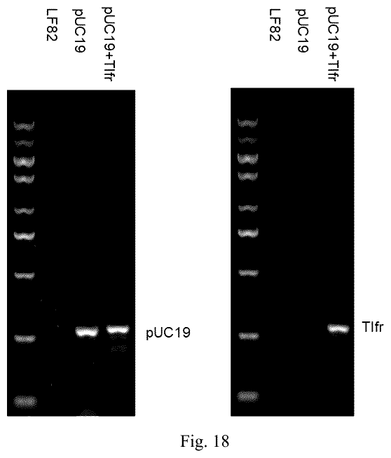

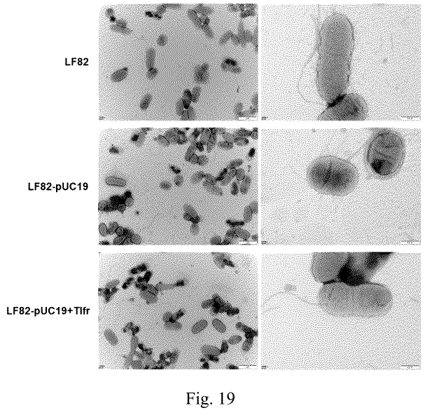

The TIfr gene was inserted into a pUC19 plasmid to construct a recombinant plasmid (as shown in FIG. 15) according to a conventional approach. The recombinant plasmid containing the TIfr gene was transformed into a LF82 strain through electrotransformation, where whether the target gene was successfully transformed was confirmed by PCR and gel electrophoresis (as shown in FIG. 18). Then the LF82 strain was cultured on an agar plate containing ampicillin to the logarithmic phase, and observed under a transmission electron microscope. Additionally, a blank control group (i.e., the wild-type LF82 strain) and a negative control group (i.e., the LF82 strain transformed with a pUC19 plasmid without the target gene) were set. As shown in FIG. 19, the fimbrial growth was clearly observed in the blank control group and the negative control group, while the LF82 strain transformed with the target gene was free of fimbriae, indicating that the exogenous expression of the TIFR protein can effectively inhibit the type I fimbriae of the LF82 strain.

In conclusion, the hypothetical protein (gene ID: GW576_23830, named TIFR) can inhibit the type-I fimbriae of E. coli.

What is claimed is:

1. A method for inhibiting growth of type I fimbriae of an Escherichia coli (E. coli) strain, comprising:
constructing a recombinant plasmid carrying a TIfr gene, wherein the TIfr gene consists of SEQ ID NO: 6; and
introducing the recombinant plasmid into the E. coli strain.

2. The method of claim 1, wherein the recombinant plasmid is a pUC19 plasmid.

3. The method of claim 1, wherein the recombinant plasmid is introduced into the E. coli strain through electrotransformation.
